(19) United States
(12) Patent Application Publication
(10) Pub. No.: US 2010/0152275 A1
(43) Pub. Date: Jun. 17, 2010

(54) Drug or Supplement Combination with Conjugated Linoleic Acid for Fat Loss in Mammals

(75) Inventors: Michael Ernest Fromm, Lincoln, NE (US); Shan Jiang, Lincoln, NE (US); Jess L. Miner, Lincoln, NE (US)

Correspondence Address: Michael Fromm, 2540 Woodscrest Ave, Lincoln, NE, 68502, US

(73) Assignee: Kaloness, Inc., Lincoln, NE (US)

(21) Appl. No.: 12/630,681

(22) Filed: Dec. 3, 2009

Related U.S. Application Data
(60) Provisional application No. 61/201,505, filed on Dec. 11, 2008.
(60) Provisional application No. 61/216,706, filed on May 20, 2009.
(60) Provisional application No. 61/184,033, filed on Jun. 4, 2009.

(51) Int. Cl. A61K 31/201; A61K 31/40; A61P 3/00; A61P 9/00

(52) U.S. Cl. 514/423; 514/560

(57) ABSTRACT

Food, feed or drug combinations with conjugated linoleic acid are described that cause enhanced fat loss in mammals more efficiently than any of the individual components of the combination. Food, feed, or drugs that activate AMP activated protein kinase, agonists of nuclear receptors that bind RXR in adipocytes, or statin inhibitors were found to be more effective for fat loss when combined with conjugated linoleic acid.

### CROSS-REFERENCE TO RELATED APPLICATIONS

Three provisional applications were filed that support the current application:

Title: Combinations with Conjugated Linoleic Acid for Enhanced Fat Loss in Mammals Ser. No. 61/201,505

### Filed: Dec. 11, 2008

Title: Method for fat loss in mammals: effective combinations using receptors Ser. No. 61/216,706

### Filed: May 20, 2009

Title: Method for fat loss in mammals: effective combinations with statins Ser. No. 61/184,033

The three inventors of the current application are also the three inventors named on each of the above provisional applications, and each of these inventors participated in making each of the inventions claimed within this application.

### STATEMENT REGARDING FEDERALLY SPONSORED RESEARCH OR DEVELOPMENT

Not applicable.

### REFERENCE TO A “SEQUENCE LISTING

Not applicable.

### BACKGROUND OF THE INVENTION

The ability to reduce body fat has been and continues to be an important goal for humans and animals. The benefits are both in appearance and improved health for humans and companion animals such as dogs and cats. In humans, obesity is considered a major health problem, with longer term consequences in cardiovascular disease, diabetes, heart attacks, insulin resistance, and metabolic syndrome. For agricultural animals, the benefit is a high quality lean meat. Despite the tremendous benefits to be obtained, the goal of being able to reduce body weight or body fat has remained elusive and is still a highly sought after goal. Considerable commercial interest exists in providing a healthy and efficient method of fat loss and this is still an unmet need.

Conjugated linoleic acid (CLA), a set of isomers of linoleic acid in which the double bond positions form a conjugated bond system, have been reported to be effect for fat loss in some mammals (U.S. Pat. No. 7,365,099), particularly in mice. The ability of CLA to cause fat loss in humans is limited and recent reviews of human trials indicate little to no weight loss (Plourde M, Jew S, Cunnane S C, Jones P J (2008) Conjugated linoleic acids: why the discrepancy between animal and human studies? Nutr Rev. 66:415-21).

A search of the USPTO for patents mentioning both CLA and metformin found six patents.

U.S. Pat. No. 7,320,972: 4-Biarylyl-1-phenylazetidin-2-ones;
U.S. Pat. No. 7,304,089: Preparation for improved dietary utilization;
U.S. Pat. No. 7,001,746: Methods and compositions for the differentiation of human preadipocytes into adipocytes;
U.S. Pat. No. 6,893,627: Method for treating type 2 diabetes with an extract of Artemisia;
U.S. Pat. No. 6,881,854: Conjugated unsaturated glyceride mixtures and a method for producing the same;
U.S. Pat. No. 6,440,931: Conjugated linoleic acid in treatment and prophylaxis of diabetes).

None of these are directly related to the current invention and do not describe or enable the use of CLA and metformin for fat loss in mammals.

### BRIEF SUMMARY OF THE INVENTION

The present invention is summarized as a method for controlling body fat level in mammals (both human and non-human) that includes the step of administering CLA and an agent that activates AMP activated protein kinase (AMPK) in an amount sufficient to control body fat in the mammal.

In a related embodiment, an agent that activates RXR or PPARβ/δ can be used in combination with CLA for reducing body fat in a mammal. In a second related embodiment, an agent that inhibits HMGCoA Reductase can be used in combination with CLA for reducing body fat in a mammal.

In yet another embodiment, the method can use specific isomers of the set of CLA isomers, including but not limited to the trans-10, cis-12 isomer of CLA, or fatty acids of different lengths that contain the trans-10, cis-12 conjugated double bond, in amounts effective to elicit a synergistic body fat when used with an agent that activates either AMPK, RXR or PPARdelta, or inhibits HMGCoA Reductase.

Other objects, features and advantages of the invention will become apparent upon consideration of the following detailed description.

### DEFINITIONS

ACC Acetyl-CoA Carboxylase
AICAR aminoimidazole carboxamide ribonucleotide is a ribonucleoside analog that is phosphorylated in the cell to form ZMP
AMP Adenosine monophosphate
AMPK AMP Activated Protein Kinase
CLA Conjugated linoleic acid. Also used more broadly to include all isomers of linoleic acid with a conjugated pi bond system. Specifically includes the trans-10, cis-12 isomer in its pure form or as part of a mixture. For the purpose of this application, the term CLA also includes fatty acids of different lengths that contain trans-n, cis-(n+2) conjugated double bond such as conjugated nonadecadienoic acid [1, 2], of lengths from C16 to C20, and n=8 to 12.
ERK Extracellular signal-regulated kinase
ISR Integrated Stress Response
JNK c-Jun N-terminal kinase
LXR Liver X Receptor, nuclear receptor family
Metformin (trade names Glucophage, Riomet, Fortamet, Glumetza, Obimet, Dianben, Diabex, Diaformin, and others).
MCP-1 Monocyte chemotactic protein-1
NF-κB Nuclear Factor Kappa B
PPARβ/γ Peroxisome proliferator-activated receptor gamma PPARβ/δ Peroxisome proliferator-activated receptor beta/delta, also referred to as Peroxisome proliferator-activated receptor beta or Peroxisome proliferator-activated receptor delta
RAR Retinoic Acid Receptor
RXR Retinoid X Receptor
WAT White adipose tissue
ZMP an AMP mimetic.

### BRIEF DESCRIPTION OF THE SEVERAL VIEWS OF THE DRAWINGS

FIG. 1. Metformin increases the response to t10c12 CLA in mice. Forty-two MC and 42 mL mice were fed diets containing soy oil or soy oil with 0.25% t10c12 CLA and drinking water containing either none, 0.33 mg/ml, or 3.33 mg/ml metformin (Met) for 14 days (n=6 mice per treatment). A positive control diet contained the standard amount of 0.5% t10c12 CLA without any metformin. Body weights were recorded and retroperitoneal (RP) and epididymal fat pads from all the mice were isolated and weighed. A) One RP fat pad is shown for each of the six mice for each of the treatments indicated for the ML genotype.

### DETAILED DESCRIPTION OF THE INVENTION

The present invention relates to a treatment to reduce fat in mammals (defined as humans and animals). It is an object of the present invention to provide a composition that causes fat loss in mammals. The present invention provides one or more compositions that can cause fat loss in mammals.

An inventive aspect is the discovery that combinations of conjugated linoleic acid (CLA), or other fatty acids containing conjugated double bonds of the configuration trans-10, cis-12 (such as calendic acid), or similar in geometry if numbered differently due to different fatty acid chain lengths, and selected molecules or compositions capable of activating AMP-activating protein kinase (AMPK) cause enhanced fat loss in mammals. The present invention recognizes compositions containing CLA and one or more compounds causing increases in AMPK activity are an effective means of causing fat loss in mammals.

An interesting and important aspect of CLA biology is its apparent cell type selectivity for lipogenic cells. CLA increases AMPK activity predominantly in adipocyte tissues. This is due in part to a selective accumulation of CLA in adipose tissue in mammals [3, 4]. This may also be due in part to a selective response to CLA in adipocytes as we have found 3T3-L1 fibroblasts do not show the robust response to CLA that adipocytes do [5]. We also have discovered that CLA only weakly affects livers in CLA fed mice, using microarray analysis. Chemical combinations of CLA and other molecules that affect signaling in cells benefit from the foundation state created by CLA which increases activated AMPK and reduces PPARγ activity. PPARγ is an important regulatory protein in adipocytes [6]. The ability of CLA to activate AMPK and reduce PPARγ activity provides a sensitivity to the effects of other molecules that allows them to act most effectively in adipocytes to reduce fat when their mechanisms complement that of CLA.

The present invention recognizes that CLA in combination with one or more chemicals that cause either additional activation of AMPK or that further reduce PPARγ activity are beneficial for increased fat loss in mammals. Most drugs or nutritional supplements generally do not show tissue specificity, which increases the chances of undesirable side effects. For example, PPARγ antagonists have been discovered that cause delipidation in adipocyte cultures [7, 8], but none have succeeded in clinical trials, presumably because of their detrimental inhibition of PPARγ in other tissues. Our data supports our premise that the response to CLA occurs most robustly in lipogenic cells, and thus provides an excellent platform for adding one or more types of additional molecules for reducing adiposity that collectively provide a more tissue specific effect. This multiple component approach benefits from the lower levels needed of each chemical component, reducing possible side effects, with the main tissue specificity provided by t10c12 CLA. Specifically, many weight loss drugs that have undesirable side effects can perform better at lower doses in the presence of CLA.

CLA is a collective name for a class of positional and structural isomers of linoleic acid that contain conjugated double bonds wherein at least one is of the cis configuration and one is of the trans configuration. The present invention relates to CLA isomers containing the trans-10, cis-12 configuration (numbering starts at the carboxyl group). This configuration may be alone or in combination with additional double bonds in the molecule such as are present in calendic acid or fatty acids containing the trans-10, cis-12 configuration that are of different length than the 18 carbons present in CLA, such as Conjugated nonadecadienoic acid [1, 2]. Additionally, the trans-10, cis-12 containing fatty acid may be contained in a mixture with other isomers of CLA such as cis-9, trans-11 linoleic acid, as well as additional C18:2 isomers or other fatty acids. Compositions containing CLA may contain additional molecules or materials suitable for consumption or injection and are not limited by the examples presented here.

CLA is taken to mean a fatty acid mixture, possibly present in a more complex composition or mixture, that contains a fatty acid content of 20%, more preferably 50%, still more preferably 60%, 70%, 80% or 90%. Most preference is given to a content of 95% or more.

CLA has been reported to have modest beneficial effects in humans. For reviews see [9-12]. The most recent review concludes CLA has no significant effects [12] when used alone. CLA exists naturally at modest amounts in various foods, particularly those from ruminants and can be prepared chemically or through biotechnology methods.

These methods of preparation are widely known to those skilled in the arts and are not considered part of the current invention.

### Peroxisome Proliferator-Activated Receptors and Retinoid X Receptor Biology

Peroxisome Proliferator-activated Receptor γ (PPARγ) is a member of the nuclear hormone receptors and is required for differentiation and maintenance of the adipocyte cell [6]. PPARγ is activated by a number of fatty ligands and is regulated by interactions with multiple coactivators or corepressors, facilitating complex modes of regulation [6]. PPARγ also modulates the inflammatory response in adipocytes. PPARγ and NE-KB are mutually antagonistic in t10c12 CLA-treated human adipocytes [13]. PPARγ antagonizes NF-κB through transrepression via SUMOylation of PPARγ and stabilization of the nuclear receptor corepressor on promoters of inflammatory genes in macrophages. PPARγ phosphorylation, degradation, or SUMOylation mechanisms presumably operate in adipocytes.

PPARγ requires binding to retinoid X receptor (RXR) to form an active functional protein complex. The PPARγ/RXR heterodimer is of the permissive class of nuclear receptors as it can be activated by ligands that bind to either PPARγ or RXR or to both. RXR is a required subunit for forming functional heterodimers with other members of the nuclear receptor superfamily such as farnesoid X receptor, liver X receptor α or β (LXRα, LXRβ), PPARα, PPARβ/δ, thyroid hormone receptor, vitamin D receptor, and members of the retinoic acid receptors (RAR).

A second aspect of the present invention is that because CLA reduces PPARγ activity, it is possible to more effectively further reduce PPARγ activity in the presence of a CLA with a second chemical that is a ligand for a nuclear receptor that forms heterodimers with RXR, a required partner for PPARγ activity. This invention can be practiced with CLA and other AMPK activating chemicals, and/or multiple ligands to the nuclear receptors, but is described for only one ligand for clarity. These ligand-activated receptors can compete with PPARγ for RXR, reducing the amount available for PPARγ, and thus reducing PPARγ activity and causing more fat loss. Adipocytes have isoforms of RAR, LXR, and PPARβ/δ nuclear receptors that are useful for this purpose, particularly RAR and PPARβ/δ receptors as LXR receptors can increase lipogenesis in the liver.

Our working model for t10c12 CLA signaling. CLA strongly activates AMPK to a phosphorylated state (p-AMPK), which initiates an integrated stress response (ISR) [5]: either step might be cell-type specific for CLA. The ISR activates the NF-κB, JNK, and ERK axis, which together initiate an inflammatory response. AMPK, NF-κB, JNK, or ERK also directly or indirectly inhibit PPARγ. Ligand activated isoforms of RAR, LXR, or PPARβ/δ can also compete with PPARγ for RXR, reducing PPARγ activity and increasing fat loss. One aspect of the present invention is that ligands that activate isoforms of RAR, LXR, or PPARβ/δ can increase fat loss in the presence of CLA. These ligands can be combined in a composition comprising (whether in a single composition or consumed separately such that these chemicals occur in the blood and fluids of the body in this combination) CLA, with or without one or more AMPK activating chemicals, or with or without one or more ligands to one or more of the isoforms of RAR, LXR, or PPARβ/δ. The important aspect is that these substances are present in the blood and fluids of the mammal, regardless of the method of delivery or distribution of the components before entering the body.

It is understood that these chemicals when used in combination with CLA do not need to be in a single physical mixture such as a pill or emulsion or solution or solid. The important feature is that these are consumed within a time period that they are present in the blood or fluids of the body at the same time or in close proximity in time, so that their biological effects are cumulative. This is called a composition from the perspective of the cumulative effects in the body, not the physical state prior to being introduced into the body. The physical mixture prior to being introduced to the body is not critical for the present invention. This invention considers separate deliveries or combined deliveries of the chemicals into the body to be equally effective and part of the composition in the blood or fluids of the body that is used to achieve fat loss.

One aspect of the present invention relates to a composition of CLA and one or more AMPK activating substances. Preferably, the second AMPK substance is a chemical or a chemical present in a mixture, such as herbal extracts, mixtures of chemicals with similar effects, or in mixtures designed for flavor or texture for more flavorful or functional consumption. The second substance could also be administered by other means such as injection or absorption and other methods known for drug delivery. The important aspect is that both CLA and a second substance are present in the blood and fluids of the mammal, regardless of the method of delivery.

In one embodiment the AMPK activating substance is a drug known to activate AMPK in mammals. This includes drugs currently is use and those being developed wherein these drugs are known to activate AMPK as a component of their therapeutic affect. Preferably the drug is from the class of biguanides drugs known to activate AMPK. Most preferably, the drug is metformin (or equivalent names for the same active chemical).

Metformin (trade names Glucophage, Riomet, Fortamet, Glumetza, Obimet, Dianben, Diabex, Diaformin, and others).

Thiazolidinediones are also known to activate AMPK but some of these have opposite affects of increasing fat gain due to their activation of the PPARγ receptor. AMPK activators of this class of drugs are available that have less activity with PPARgamma and are preferred for the present invention [14].

Other AMPK activating substances that are useful in the present invention in combination with CLA, with or without metformin: Berberine, quercitin, Leptin, adiponectin, Genistein, green tea and epigallocatechin-3-gallate (EGCG), capsaicin, beta-sitosterol, Alpha-lipoic acid (ALA), a naturally occurring antioxidant, Isoproterenol (isoprenaline), a synthetic structural isomer of dihydrocapsiate, isodihydrocapsiate (8-methylnonanoic acid 3-hydroxy-4-methoxy benzyl ester), black tea and theaflavins, with bisphenol A diglycidyl ether (BADGE) or caffeine, MEDICA fatty acid analogs, a new drug activator of AMPK, namely A769622, caffeic acid phenethyl ester, cucurbitane triterpenoids.

### EXAMPLES

### Example 1

### CLA Activates AMPK

Mouse 3T3-L1 cells in tissue culture were differentiated in adipocytes using published methods [5]. The level of phosphorylated AMPK and acetyl CoA carboxylase (ACC), one of AMPK's known in vivo substrates, was measured at various times after treating 3T3-L1 adipocytes with either 100 uM linoleic acid or trans-10, cis-12 CLA (t10c12 CLA). T10c12 CLA-treated adipocytes show increased levels of both phosphorylated AMPK and phosphorylated ACC as early as 30 min after exposure and this enhanced phosphorylation level persisted for the 16 hr duration of the experiment. The total amounts of AMPK and ACC were also determined with antibodies that recognize the respective proteins regardless of phosphorylation status. The levels of both proteins remained constant during the experiment. These results indicate AMPK is activated early and persistently during exposure to t10c12 CLA. (Data in published form in [15] (Jiang S, Wang Z, Riethoven J J, Xia Y, Miner J, Fromm M (2009) Conjugated Linoleic Acid Activates AMP-Activated Protein Kinase and Reduces Adiposity More Effectively When Used with Metformin in Mice. J. Nutr. 139:2244-2251, which is included in its entirety by reference herein).

### Example 2

### AMPK Activity is Required for CLA-Mediated Fat Loss

Compound C is a potent inhibitor of AMPK and was used to investigate whether AMPK is required for the t10c12 CLA-mediated delipidation response. 3T3-L1 adipocytes were treated with compound C in combination with either control linoleic acid (LA) or t10c12 CLA. Triglyceride levels from samples treated with either of these combinations were similar to the LA control levels. Treatment with 10c12 CLA alone produced a 50% reduction in triglyceride levels. These results indicate compound C is inhibiting a protein kinase, most likely AMPK, necessary for t10c12 CLA-mediated delipidation. We demonstrated by western blot that phosphorylation of AMPK and its in vivo substrate Acetyl-Coenzyme A Carboxylase (ACC) are inhibited, demonstrating AMPK was inhibited by compound C in these experiments. (Data in published form in [15] (Jiang S, Wang Z, Riethoven J J, Xia Y, Miner J, Fromm M (2009) Conjugated Linoleic Acid Activates AMP-Activated Protein Kinase and Reduces Adiposity More Effectively When Used with Metformin in Mice. J. Nutr. 139:2244-2251, which is included in its entirety by reference herein).

### Example 3

### CLA in Combination with an AMPK Activator Causes More Fat Loss

Two well known AMPK activators with very different chemical structures are metformin and AICAR. Metformin is from the class of biguanides while AICAR is a ribonucleoside analog that is phosphorylated in the cell to form ZMP, an AMP mimetic. The individual effects of metformin and AICAR alone or when combined with t10c12 CLA on triglyceride levels were examined after treating 3T3-L1 adipocytes with these chemicals. A lower 50 μM level of t10c12 CLA was used in the experiment to better measure any effects of the combinations. Individual treatments with either 50 μM t10c12 CLA, metformin, or AICAR showed triglyceride reductions of 16 to 22% when compared to the control LA treatments. The combined treatments of either metformin or AICAR with 50 μM t10c12 CLA reduced triglyceride levels by 48 to 50%. This demonstrates the combination of CLA with an AMPK activator provides more fat loss than either chemical alone in the same conditions, and more than expected from additive effects. (Data in published form in [15] (Jiang S, Wang Z, Riethoven J J, Xia Y, Miner J, Fromm M (2009) Conjugated Linoleic Acid Activates AMP-Activated Protein Kinase and Reduces Adiposity More Effectively When Used with Metformin in Mice. J. Nutr. 139:2244-2251, which is included in its entirety by reference herein).

### Example 4

### CLA in Combination with an AMPK Activator Causes More AMPK Activation

The phosphorylation levels of AMPK and ACC were then determined after treating 3T3-L1 adipocytes with either metformin, AICAR, or t10c12 CLA alone or in combination. Each chemical increased the phosphorylation of these proteins after 2 or 8 hr of treatment. The combination treatments showed higher levels of phosphorylated AMPK and ACC than those observed with metformin or AICAR (Table 1) and in Jiang et al, 2009 [15]. The combined treatments produced phosphorylation levels similar but higher than those of t10c2 CLA alone. A 2-way ANOVA statistical analysis found this was a positive interaction and more than an additive effect [15].

TABLE 1
AICAR, metformin, or CLA treated adipocytes have
increased phosphorylation of AMPK and ACC

| | Phospho-AMPK | | Phospho-ACC amount | |
|---|---|---|---|---|
| | Amount | | Amount | |
| Treatment | 2 hr | 8 hr | 2 hr | 8 hr |
| LA | 1 | 1 | 1 | 1 |
| CLA | 6.0 | 1.7 | 4.1 | 3.3 |
| LA & AICAR | 3.7 | 0.7 | 3.5 | 1.1 |
| CLA & AICAR | 7.5 | 1.8 | 4.7 | 3.2 |
| LA & Metformin | 4.2 | 1.2 | 3.8 | 1.6 |
| CLA & Metformin | 11.1 | 2.1 | 6.1 | 3.3 |

3T3-L1 adipocytes were treated with the indicated compounds and the amount of phosphorylated or total AMPK or ACC was measured by western blot analysis.

### Example 5

### AMPK Activators Reduce CLA-Mediated Inflammation

CLA has been reported to cause inflammation [5, 16] which is considered a non-healthy condition. Unexpectedly, we found AMPK activators reduced the CLA-mediated inflammatory response while increasing the fat loss (see example 3 for fat loss). Mouse 3T3-L1 cells in tissue culture were differentiated in adipocytes using published methods. Metformin or AICAR was used in combination with t10c12 CLA in the media containing the adipocytes for 12 hrs. RNA was isolated and the inflammatory response, as measured by MCP-1 (an inflammatory cytokine) mRNA levels, was determined by real time PCR of the reverse transcribed RNA. These results are shown in Table 2. CLA treatment induced the inflammatory MCP-1 23 fold relative to the LA control while neither metformin nor AICAR alone produced a significant induction of MCP-1 mRNA. The combined treatments with CLA showed less inflammation than CLA alone, while showing stronger delipidation (example 3). This indicates the combination treatments can produce a stronger delipidation effect with a lower inflammatory response than can be obtained with t10c12 CLA alone. This provides an unexpected benefit to the combination as inflammation is considered an undesirable condition for optimum health in mammals and humans.

TABLE 2
Inflammatory response to individual and
combination treatments in adipocytes.

| Treatment | MCP-1 mRNA levels |
|---|---|
| LA | 1 |
| CLA | 23 |
| LA & AICAR | 1 |
| CLA & AICAR | 9 |
| LA & Metformin | 0.5 |
| CLA & Metformin | 17 |

3T3-L1 adipocytes were treated with the indicated compounds and the amount of MCP-1 mRNA, an inflammatory marker, was measured by real time PCR.

### Example 6

Human patents will be given a combination of t10 c12 CLA and metformin daily for a period of one to three months. The daily doses are described below. Body fat measurements will be taken initially, and at two, four and twelve weeks. Patents will be asked to consume their normal food amounts and types.

Results: Patients will be found to have a significant fat loss compared to their starting weight in this experiment.

Daily dose of CLA. 0.001 to 1 g/kg of bodyweight, preferably 0.001 g to 0.5 g/kg daily, more preferably 0.01 to 0.5 g/kg of CLA (either as a mixture or enriched for trans-10, cis-12 CLA).

Daily dose of metformin. In combination with CLA at the above doses, metformin (or an equivalent name for the same chemical) is used at the therapeutically prescribed doses known to medical personnel and those skilled in the art for the treatment of insulin resistance, diabetes and metabolic syndrome. Typical metformin doses range from 0.5 to 2.5 g per day, with the larger amounts split into several smaller tablets to be taken at intervals such as meals or before bedtime.

### Example 7

### Microarray Analysis of t10c12 CLA Activated Pathways

Recent microarray analyses of the WAT of mice fed t10c12 CLA by our laboratory [16] and others found three major trends. First, most of the key adipocyte lipid-related genes show reduced expression, both in the biosynthetic pathways and in the lipases involved in the export of fatty acids from adipocytes. The transcripts and/or proteins of the major adipocyte transcription factors C/EBPα, PPARγ, and SREBP1c are also strongly reduced. Second, the first several days of dietary t10c12 CLA induce an inflammatory response of cyclo-oxygenase 2 (Cox2); numerous cytokines including interleukins 1, 6, 10, 15, and 17; many of their receptors; and more than 30 members of the CXC or CC ligand family of cytokines [16]. This initial inflammatory response is transient: the expression levels of many of these genes returns to normal levels following the first week of treatment, and the remaining genes are expressed at lower levels than the initial response. Third, key genes involved in fatty acid oxidation show increased mRNA levels. In particular, carnitine palmitoyltransferase I (CPT1), uncoupling protein 1 (UCP1), and uncoupling protein 2 (UCP2) show increases [5, 16]. The response in UCP1 and UCP2 genes is quite strong, as the levels of the induced mRNAs are in the 95^{th} and 90^{th} percentiles of the most abundant mRNAs in the adipocyte, respectively [16]. UCP1 is usually exclusively expressed in brown adipose tissue, so this is a remarkable change in expression pattern with likely physiological effects. There is not a consensus that increased metabolic heat output occurs in t10c12 CLA treatments, but this increased metabolism has been observed in some studies [17].

### Example 8

### Metformin, t10c12 CLA, or the Combination Produce Similar Microarray Profiles

We further defined the responses to t10c12 CLA by comparing the microarray-measured gene expression changes that occur during treatments of 3T3-L1 adipocytes with control linoleic acid (LA), t10c12 CLA, metformin or a metformin-t10c12 CLA combination. The purpose of the experiment was to determine whether new classes of genes were activated or whether the responses were fairly similar, the latter situation was found. This is most apparent from the correlation coefficients of the gene expression changes for the various treatments (Table 3). The metformin or t10c12 CLA treatments look very similar to each other and to the combined treatment (Table 3). This similarity is even more striking when comparing the changes in the specific ISR or lipid pathways which we previously found showed definitive behaviors in response to t10c12 CLA [5]. We found the expression of the genes in the predefined ISR or lipid pathways had correlation coefficients of 0.95 or better (Table 3).

The conclusion from our microarray analysis is that the gene expression responses are very similar except that t10c12 CLA induces expression of a set of inflammatory genes that are not as highly induced in the metformin treatment. Specifically, metformin treatment produced lower levels of induction for the CD14 antigen (CD14), cardiotrophin-like cytokine factor 1 (Clcf1), cyclo-oxygenase 2 (Cox2), colony stimulating factor 1 (Csf1), MCP-1, oncostatin M receptor (Osmr), plasminogen activator inhibitor type 1 (PAI or Serpine1), and suppressor of cytokine signaling 3 (SOCS3) genes that respond more strongly with t10c12 CLA treatments (data not shown). See Jiang et al., 2009 for a more complete description of the published results [15].

TABLE 3
Correlation coefficients for gene expression changes in treatments
containing t10c12 CLA, metformin or the combination*

| | Metformin | t10c12 CLA + Metformin |
|---|---|---|
| All significant genes | | |
| t10c12 CLA | 0.75 (n = 246) | 0.86 (n = 410) |
| t10c12 CLA + Metformin | 0.92 (n = 408) | |
| ISR genes (n = 63) | | |
| t10c12 CLA | 0.91 | 0.96 |
| t10c12 CLA + Metformin | 0.97 | |
| Lipogenesis (n = 69) | | |
| t10c12 CLA | 0.95 | 0.97 |
| t10c12 CLA + Metformin | 0.98 | |

*Each treatment contained three replicas. The number of genes being compared is represented by n. For the ‘all significant genes’ the gene list is derived from the union of genes in either treatment that show a four fold change in expression and a Benjamini-Hochberg-adjusted p-value <=0.1 in at least one of the treatments being compared. The ISR and lipogenesis gene lists were derived from a list of genes previously found to be responsive to t10c12 CLA or tunicamycin in 3T3-L1 adipocytes [5]. The Spearman correlation was used with associated p values <= 2.2e−²⁴.

### Example 9

### Phenformin Produces an Inflammatory and Delipidation Response

Phenformin is similar in structure to metformin but is a more potent AMPK activator on a molar concentration basis and on a tissue toxicity basis. We found phenformin results in higher p-AMPK levels and, unlike metformin, is sufficient for the type of strong delipidation observed with t10c12 CLA treatments. Phenformin also has an inflammatory response that is stronger than that observed with metformin. These results support our model that strong AMPK activation in adipocytes is sufficient for a robust delipidation response. This implies t10c12 CLA only needs to activate AMPK in a tissue-specific manner to account for the majority of its effects. However, because phenformin can cause toxic lactic acidosis [18], it is less desirable than metformin for use in humans and animals. These results support our hypothesis that stronger AMPK activation facilitates fat loss, and points out the need for proper and effective chemical targeting to the target tissues in vivo, i.e., adipose tissue. The ability of CLA to preferentially activate AMPK in adipose tissue provides much of this biological targeting, and thereby enhances the effects of less specific drugs in the adipose tissue as well.

### Example 10

### T10c12 CLA is Selective for Adipocytes

We have microarray analyses of 3T3-L1 fibroblasts prior to differentiation and from liver tissues in mice treated with t10c12 CLA that support the hypothesis that t10c12 CLA is selective in the types of cells responding. The ISR is a cell response that most cells are capable of inducing and is a key part of the response to t10c12 CLA [5, 19]. We found that 3T3-L1 fibroblasts do not show this response when treated with t10c12 CLA [5]. More recently, we compared the response during differentiation of 3T3-L1 fibroblasts into adipocytes and found that t10c12 CLA could only induce an ISR after several days of differentiation, when the cells appear to have adipocyte properties such as small oil droplets. Our microarray analysis of liver tissues of mice treated with t10c12 CLA showed a weak ISR that was much weaker than that observed in adipose tissue, and lacked a significant delipidation or inflammatory response. This data supports CLA showing a preferential or selective effect in adipose tissue.

### Example 11

### Metformin and t10c12 CLA Responses are Additive in Mouse Trials

Two genotypes of mice with differences in metabolic rates, the metabolism control (MC), and metabolism low (ML) genotypes, were analyzed for their response to diets containing various combinations of soy oil, 0.25% dietary t10c12 CLA, and metformin or control diet for two weeks (these results are in published form in [15]). We found metformin could potentiate the t10c12 CLA effect in mice. The MC genotype lost too much fat on the 0.25% t10c12 CLA to evaluate the interactions with metformin, except for changes in body weight, where MC mice given 0.25% CLA and metformin weighed less than mice given only 0.25% CLA (Table 4).

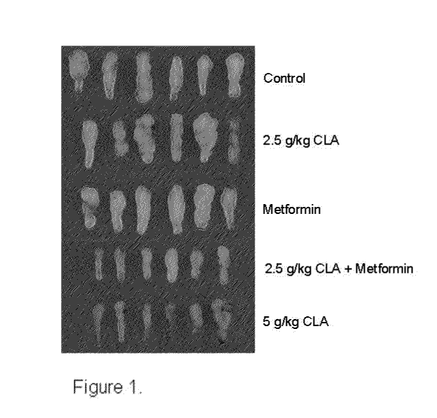

ML mice consuming 0.25% t10c12 CLA at either a low or high dose of metformin showed reductions in WAT weights (FIG. 1: row 4 labeled 2.5 g/kg CLA+Met) similar to those of the higher 0.5% t10c12 CLA dose (FIG. 1: row 5 labeled 5 g/kg CLA), and these reductions in WAT were significantly more than the 0.25% t10c12 CLA (FIG. 1: row 2 labeled 2.5 g/kg CLA) or metformin control treatments (FIG. 1A: row 3 labeled Metformin) or control untreated mice (FIG. 1: row 1 labeled Control). The ranges of the individual weights of the retroperitoneal fat pads or the epididymal fat pads are shown in Table 3. Note that metformin alone in ML mice increased fat pad weights relative to control untreated mice, while 0.25% CLA had no significant change from ML control mice, while the combination of 0.25% CLA and either dose of metformin showed significant fat loss in ML mice. This result would not be expected from the individual effects of CLA or metformin. These in vivo studies in mice confirm the observations in the 3T3-L1 adipocyte system and again support the premise that the 3T3-L1 adipocyte system is a reasonably good model system for mouse WAT. The combination of CLA and metformin provides several benefits. Less CLA is needed for fat loss, which reduces the amount of high caloric fat (CLA) needed for weight loss. This is due to the positive interaction of CLA and metformin. Also, as described above, metformin reduces the inflammatory response initiated by CLA while still providing the fat reducing benefits. The combination is also more efficient at creating fat loss than either compound alone in other mammals.

TABLE 4
The effect of combining metformin and t10c12 CLA on lipid toss in ML and MC mice^{1,2}

| Met0³ | | Met2 | | Met20 | | Significance | | |
|---|---|---|---|---|---|---|---|---|
| −CLA | +CLA | −CLA | +CLA | −CLA | +CLA | Met | CLA | Met × CLA |

ML mice

| | | | | | | | | | |
|---|---|---|---|---|---|---|---|---|---|
| BW | 2.46 ± 0.48^{a} | 1.89 ± 0.95^{ab} | 2.89 ± 1.00^{a} | 0.68 ± 0.40^{b} | 2.54 ± 0.51^{a} | 0.88 ± 0.48^{b} | 0.07 | 0.01 | 0.50 |
| EPI | 0.90 ± 0.09^{ab} | 0.89 ± 0.17^{ab} | 1.06 ± 0.13^{a} | 0.71 ± 0.10^{bc} | 1.08 ± 0.12^{a} | 0.64 ± 0.07^{c} | <0.01 | <0.01 | <0.01 |
| RP | 0.28 ± 0.05^{a} | 0.27 ± 0.08^{ab} | 0.37 ± 0.10^{a} | 0.19 ± 0.04^{bc} | 0.33 ± 0.06^{a} | 0.15 ± 0.02^{c} | 0.76 | 0.02 | 0.34 |

MC mice

| | | | | | | | | | |
|---|---|---|---|---|---|---|---|---|---|
| BW | 1.51 ± 0.53^{a} | 1.42 ± 0.46^{ab} | 1.96 ± 0.90^{a} | 0.27 ± 0.48^{b} | 2.82 ± 0.96^{a} | 0.47 ± 0.96^{b} | 0.77 | 0.03 | 0.31 |
| EPI | 0.64 ± 0.11^{a} | 0.31 ± 0.07^{b} | 0.59 ± 0.07^{a} | 0.36 ± 0.11^{ab} | 0.48 ± 0.09^{a} | 0.29 ± 0.04^{b} | 0.48 | <0.01 | 0.72 |
| RP | .28 ± 0.05^{a} | 0.10 ± 0.01^{b} | 0.23 ± 0.04^{a} | 0.10 ± 0.04^{b} | 0.23 ± 0.06^{a} | 0.06 ± 0.01^{b} | 0.48 | <0.01 | 0.77 |

¹Data are presented as mean ± SEM, n = 6. Means in a row not sharing a common superscript differ, P < 0.1.
²The change in body weight (BW) or the final weight of the epididymal (EPI) or retroperitoneal (RP) was measured after 14 d of treatments.
³Mice were fed diets supplemented with soy oil (−CLA), or t10c12 CLA (+CLA), and given drinking water lacking (Met0) or containing metformin at 2 (Met2) or 20 mmol/L (Met20) for 14 d.

### Example 12

### Vitamin A and its Metabolites Cause Delipidation Only in Combination with t10c12 CLA

The retinoid family of vitamin A (retinol), retinaldehyde, retinoic acid, and vitamin A precursor β-carotene, have complex effects in adipocytes. Vitamin A supplementation decreases adipose mass in rats [20]. Retinaldehyde levels inversely correlate with adiposity levels in mice, the expression of PPARγ-regulated genes was reduced in its presence, and mice deficient in metabolizing retinaldehyde are resistant to diet induced obesity [21]. All trans retinoic acid (ATRA) stimulates lipogenesis in mature adipocytes, but can inhibit differentiation of preadipocytes into adipocytes.

All trans retinoic acid (ATRA) is a ligand for RARα, RARγ, and PPARβ/δ [22]. Cellular retinol-binding proteins (CRBP), cellular RA-binding proteins (CRABP), and fatty acid binding proteins (FABP) transport vitamin A and its metabolites to these nuclear receptors. CRBP and CRABP transport retinoids to RAR, while FABP5 transports retinoids to PPARβ/δ [23, 24]. The ratio of CRABP and FABP5 affects whether cells respond to retinoic acid predominantly with RAR or PPARβ/δ [24]. CRBP is expressed in 3T3-L1 adipocytes, indicating retinol or retinaldehyde could be targeted to RARα or RARγ. 3T3-L1 adipocytes show undetectable levels of CRABP 1 and 2 mRNA in our microarray analysis, suggesting limited targeting of retinoic acid to RARα or RARγ. The very high levels of FABP5 in our microarray analysis suggest retinoic acid is likely to be transported to PPARβ/δ in 3T3-L1 adipocytes.

Experimentally, we found that differentiated 3T3-L1 adipocytes responded to ATRA alone with increased amounts of TG, but a combination of ATRA and t10c12 CLA reduced TG levels to below those found with t10c12 CLA alone. Similarly, we found vitamin A alone had little impact of TG levels, but could increase the TG loss when combined with t10c12 CLA. We recognize the complex medical effects and dangers of ATRA, retinoids, or rexinoids, has limited their therapeutic use in humans. Longer term, any medical implementation in humans would likely be done close medical supervision for use of retinaldehyde, retinoic acid, or retinoids that bind the RAR receptor but not the RXR receptor (defined as the class of chemicals chemically related to vitamin A and having retinoic acid like biological activity).

The ability of both RAR and PPARβ/δ to respond to vitamin A or its metabolites provides two distinct mechanisms to potentiate the delipidation effects of t10c12 CLA. Both RAR and PPARβ/δ proteins can compete for RXR. PPARβ/δ has additional signaling capabilities that increase fatty acid oxidation, energy metabolism, and confers resistance to diet-induced obesity [25].

### Example 13

PPARβ/δ agonists provide another method to increase fat loss in combination with CLA. We tested the role of PPARβ/δ with the use of GW0742, a PPARβ/δ agonist. GW0742 treatment alone had little effect on TG levels, but increased TG loss when combined with t10c12 CLA, indicating a PPARβ/δ agonist alone has little effect at these concentrations, but is capable of stimulating TG loss in the presence of t10c12 CLA. We also confirmed this result in mice, where we found the combination of CLA and GW0742 caused more fat loss than CLA alone.

Additional PPARβ/δ ligand agonists include but are not limited to:

Compound 7 ([4-[3,3-bis-(4-bromo-phenyl)-allylthio]-2-chloro-phenoxy]-acetic acid) (J Med Chem. 2007 Apr. 5; 50(7):1495-503), phenoxyacetic acid derivatives GW501516 and GW0742 (GlaxoSmithKline, Brentford, UK), L165041 and L783483 (Merck, Whitehouse Station, N.J., USA), MBX-8025 (Metabolex Inc, Calif, USA), CER-002 (Cerenis Therapeutics, Mich, USA), or KD3010 (Kalypsys, Calif, USA).

### Example 14

### Hydroxycholesterols are LXR Ligands that Increase Delipidation in CLA Treated Adipocytes

Our microarray data indicate LXRα and LXRγ mRNAs are abundant in 3T3-L1 adipocytes. Activating ligands include hydroxysterols such as 24(S)-Hydroxycholesterol or 24(S),25-epoxycholesterol. We found that either of these hydroxycholesterols alone did not reduce TG levels, but did reduce TG levels when combined with t10c12 CLA.

In summary of examples 12-14, our data shows each of these ligands has no significant delipidation effects on mature adipocytes, which contradicts some published findings, cited above. Taken together, the independent ability of multiple different ligands of RAR, PPARβ/δ, or LXR to reduce TG levels only when combined with t10c12 CLA, indicates competition for RXR is a general mechanism for reducing TG levels in the presence of CLA. Other ligands binding these nuclear receptors should also be beneficial for weight loss in combination with CLA. CLA is used in combination to increase the tissue specificity of the effect of a second chemical, or of more than one additional chemicals, that collectively increase AMPK activity and/or decrease PPARγ activity in adipocytes. There is no expressed limit on the number of chemicals that can be so combined in the blood or body fluids of human or animals, but the minimum number is two, comprising CLA and a second compound. In the intact animal or human, LXR ligands generally increase fat biosynthesis in the liver and triglyceride levels in the blood or serum, which is disadvantageous. They are beneficial in the adipocyte cells as a valuable proof of principle in that context. We expect that the physiological effects of RAR and PPARβ/δ ligands in animals and humans are more beneficial for the present invention of fat loss in humans and animals.

### Example 15

### Mevastatin Increases Fat Loss in the Presence of a Low Dose of t10c12 CLA

The class of chemicals that inhibit HMG-CoA reductase, the rate limiting step in the biosynthesis of cholesterol, is often called the statins, and often have names ending in statin.

Examples of statins include but are not limited to the following:
Simvastatin
Atorvastatin
Pravastatin
Rosuvastatin
Pitavastatin
Lovastatin

| Statin | Brand name | Derivation |
|---|---|---|
| Atorvastatin | Lipitor, Torvast | Synthetic |
| Cerivastatin | Lipobay, Baycol. | (Withdrawn from the market in August, 2001 due |
| | | to risk of serious Rhabdomyolysis) Synthetic |
| Fluvastatin | Lescol, Lescol XL | Synthetic |
| Lovastatin | Mevacor, Altocor, Altoprev | Fermentation-derived |
| Mevastatin | — | Naturally-occurring compound. Found in red yeast rice. |
| Pitavastatin | Livalo, Pitava | Synthetic |
| Pravastatin | Pravachol, Selektine, Lipostat | Fermentation-derived |
| Rosuvastatin | Crestor | Synthetic |
| Simvastatin | Zocor, Lipex | Fermentation-derived. (Simvastatin is a |
| | | synthetic derivate of a fermentation product) |
| Simvastatin + Ezetimibe | Vytorin | Combination therapy |
| Lovastatin + | Advicor | Combination therapy |
| Niacin extended-release | | |
| Atorvastatin + | Caduet | Combination therapy - Cholesterol + Blood Pressure |
| Amlodipine Besylate | | |
| Simvastatin + | Simcor | Combination therapy |
| Niacin extended-release | | |

Statins are used medically to lower cholesterol levels in patients, and are not generally regarded as drugs for weight loss. Inhibition of HMG CoA-reductase also reduces the pool of isoprenoids, one of the uses of which is to postranslationally modify proteins. Through this mechanism statins can affect other biological functions. In some instances statins have been shown to activate AMPK. We tested the ability of a statin, mevastatin, to cause fat loss in the presence of CLA in 3T3-L1 adipocytes.

We treated 3T3-L1 adipocytes with control or CLA fatty acids, with or without a statin drug. We found that relative to the linoleic acid control, mevastatin alone reduced triglyceride levels by 10%. CLA alone (50 uM) reduced triglyceride levels by 25%. Mevastatin and 50 uM CLA reduced triglyceride levels to 50%, which is the maximum fat loss in this assay. This result demonstrates a combination of a statin and CLA can synergistically increase fat loss, more than what would be expected if the effects were additive. We also confirmed this result in mice trials, where the combination of CLA and atorvastatin (Lipitor) was more efficient at fat loss in 2 weeks than CLA alone. These results indicate a combination of a statin and CLA is useful for fat loss in mammals.

### Example 16

Human patents will be given a combination of t10 c12 CLA and a medically approved statin, such as Lipitor, daily for a period of one to three months. The daily doses are described below. Body fat measurements will be taken initially, and at two, four and twelve weeks. Patents will be asked to consume their normal food amounts and types.

Results: Patients will be found to have a significant fat loss compared to their starting weight in this experiment.

Daily dose of CLA. 0.001 to 1 g/kg of bodyweight, preferably 0.001 g to 0.5 g/kg daily, more preferably 0.01 to 0.5 g/kg of CLA (either as a mixture or enriched for trans-10, cis-12 CLA).

Daily dose of Lipitor. In combination with CLA at the above doses, Lipitor (or an equivalent name for the same chemical) is used at the therapeutically prescribed doses known to medical personnel and those skilled in the medical art. Typical Lipitor doses range from 0.01 to 0.02 g per day, but can be used as high as 0.08 g/day. Preferably, the Lipitor dose is in the 0.01 to 0.08 g/day range.

### REFERENCES

1. Park Y (2009) Conjugated nonadecadienoic acid is more potent than conjugated linoleic acid on body fat reduction. J Nutr Biochem.
2. Park Y, Pariza M W (2001) The effects of dietary conjugated nonadecadienoic acid on body composition in mice. Biochim Biophys Acta. 1533:171-4
3. Alasnier C, Berdeaux O, Chardigny J M, Sebedio J L (2002) Fatty acid composition and conjugated linoleic acid content of different tissues in rats fed individual conjugated linoleic acid isomers given as triacylglycerols small star, filled. J Nutr Biochem. 13:337-45
4. Yamasaki M, Mansho K, Mishima H, Kimura G, Sasaki M, Kasai M, Tachibana H, Yamada K (2000) Effect of dietary conjugated linoleic acid on lipid peroxidation and histological change in rat liver tissues. J Agric Food Chem. 48:6367-71
5. LaRosa P C, Riethoven J J, Chen H, Xia Y, Zhou Y, Chen M, Miner J, Fromm M E (2007) Trans-10, cis-12 conjugated linoleic acid activates the integrated stress response pathway in adipocytes. Physiol Genomics. 31:544-53
6. Tontonoz P, Spiegelman B M (2008) Fat and beyond: the diverse biology of PPARgamma. Annu Rev Biochem. 77:289-312
7. Lee G, Elwood F, McNally J, Weiszmann J, Lindstrom M, Amaral K, Nakamura M, Miao S, Cao P, Learned R M, Chen J L, Li Y (2002) T0070907, a selective ligand for peroxisome proliferator-activated receptor gamma, functions as an antagonist of biochemical and cellular activities. J Biol Chem. 277:19649-57
8. Miller J R, Siripurkpong P, Hawes J, Majdalawieh A, Ro H S, McLeod R S (2008) The trans-10, cis-12 isomer of conjugated linoleic acid decreases adiponectin assembly by PPARgamma-dependent and PPARgamma-independent mechanisms. J Lipid Res. 49:550-62
9. Li J J, Huang C J, Xie D (2008) Anti-obesity effects of conjugated linoleic acid, docosahexaenoic acid, and eicosapentaenoic acid. Mol Nutr Food Res. 52:631-45
10. Park Y, Albright K J, Liu W, Storkson J M, Cook M E, Pariza M W (1997) Effect of conjugated linoleic acid on body composition in mice. Lipids. 32:853-8
11. Park Y, Storkson J M, Albright K J, Liu W, Pariza M W (1999) Evidence that the trans-10,cis-12 isomer of conjugated linoleic acid induces body composition changes in mice. Lipids. 34:235-41
12. Plourde M, Jew S, Cunnane S C, Jones P J (2008) Conjugated linoleic acids: why the discrepancy between animal and human studies? Nutr Rev. 66:415-21
13. Chung S, Brown J M, Provo J N, Hopkins R, McIntosh MK (2005) Conjugated linoleic acid promotes human adipocyte insulin resistance through NFkappaB-dependent cytokine production. J Biol Chem. 280:38445-56
14. Fukui Y, Masui S, Osada S, Umesono K, Motojima K (2000) A new thiazolidinedione, NC-2100, which is a weak PPAR-gamma activator, exhibits potent antidiabetic effects and induces uncoupling protein 1 in white adipose tissue of KKAy obese mice. Diabetes. 49:759-67
15. Jiang S, Wang Z, Riethoven J J, Xia Y, Miner J, Fromm M (2009) Conjugated Linoleic Acid Activates AMP-Activated Protein Kinase and Reduces Adiposity More Effectively When Used with Metformin in Mice. J Nutr. 139:2244-2251
16. LaRosa P C, Miner J, Xia Y, Zhou Y, Kachman S, Fromm M E (2006) Trans-10, cis-12 conjugated linoleic acid causes inflammation and delipidation of white adipose tissue in mice: a microarray and histological analysis. Physiol Genomics. 27:282-94
17. House R L, Cassady J P, Eisen E J, McIntosh M K, Odle J (2005) Conjugated linoleic acid evokes de-lipidation through the regulation of genes controlling lipid metabolism in adipose and liver tissue. Obes Rev. 6:247-58
18. Luft D, Schmulling R M, Eggstein M (1978) Lactic acidosis in biguanide-treated diabetics: a review of 330 cases. Diabetologia. 14:75-87
19. Kennedy A, Overman A, Lapoint K, Hopkins R, West T, Chuang C C, Martinez K, Bell D, McIntosh M (2009) Conjugated linoleic acid-mediated inflammation and insulin resistance in human adipocytes are attenuated by resveratrol. J Lipid Res. 50:225-32
20. Jeyakumar S M, Vajreswari A, Sesikeran B, Giridharan N V (2005) Vitamin A supplementation induces adipose tissue loss through apoptosis in lean but not in obese rats of the WNIN/Ob strain. J Mol Endocrinol. 35:391-8
21. Ziouzenkova O, Orasanu G, Sharlach M, Akiyama T E, Berger J P, Viereck J, Hamilton J A, Tang G, Dolnikowski G G, Vogel S, Duester G, Plutzky J (2007) Retinaldehyde represses adipogenesis and diet-induced obesity. Nat Med. 13:695-702
22. Berry D C, Noy N (2009) All-trans-retinoic acid represses obesity and insulin resistance by activating both PPAR{beta}/{delta} and RAR. Mol Cell Biol.
23. Schug T T, Berry D C, Shaw N S, Travis S N, Noy N (2007) Opposing effects of retinoic acid on cell growth result from alternate activation of two different nuclear receptors. Cell. 129:723-33
24. Berry D C, Noy N (2007) Is PPARbeta/delta a Retinoid Receptor? PPAR Res. 2007:73256
25. Wang Y X, Lee C H, Tiep S, Yu R T, Ham J, Kang H, Evans R M (2003) Peroxisome-proliferator-activated receptor delta activates fat metabolism to prevent obesity. Cell. 113:159-70

We claim:

1. A method of increasing weight loss by providing to humans or animals CLA in combination with a second agent, wherein the second agent activates AMPK.

2. A method of increasing weight loss by providing to humans or animals CLA in combination with a second agent, wherein the second agent is a ligand for RAR or PPARbeta/delta.

3. A method of weight loss by providing to humans or animals CLA in combination with a second agent, wherein the second agent is a statin.
